(19) United States
(12) Patent Application Publication
(10) Pub. No.: US 2017/0080182 A1
(43) Pub. Date: Mar. 23, 2017

(54) Bilateral Frontal Sinus Device

(71) Applicant: Brian William Rotenberg, London (CA)

(72) Inventor: Brian William Rotenberg, London (CA)

(21) Appl. No.: 15/231,982

(22) Filed: Aug. 9, 2016

Related U.S. Application Data
(60) Provisional application No. 62/222,247, filed on Sep. 23, 2015.

(51) Int. Cl. A61M 25/00; A61M 3/02

(52) U.S. Cl. CPC A61M 25/0041; A61M 25/0097; A61M 25/0043; A61M 3/0279; A61M 25/01

(57) ABSTRACT

A bilateral frontal sinus insert which has a flexible body defining an interstitial member adjacent a horizontal tube and a vertical tube. The flexible body having entry and exits apertures for fluids and or particulates in fluid form. The fluids or particulates can include medicines. The body flexes for insertion within the sinus area, positioning within the sinus area and for extraction from the sinus area. The interstitial member is defined by an aperture or by a layer of flexible material in between the vertical and horizontal tubes. Downward or upward movement of the vertical tube is used to reduce or increase the width between the interstitial members for retaining or releasing fluid in a controlled manner.

This is a non-provisional utility patent application which claims priority to copending provisional application Ser. No. 62/222,247 filed Sep. 23, 2015, which is incorporated herein for all purposes.

### TECHNICAL FIELD

The technical field of the invention relates to a bilateral frontal sinus device, system and use thereof for controlled use of medications and/or maintaining fluid flow during and after irrigation of the entire frontal sinus cavity and releasably retaining the device in the functional position.

### BACKGROUND OF THE INVENTION

Frontal sinus surgery is one of the most challenging areas of sinus surgery due to the difficult visualization and complex anatomy of patients. The ultimate success or failure of endonasal frontal sinus surgical procedures is determined essentially by the rate of restenosis of the frontal sinus outflow tract. It is known that long-term stenting for a period of several months reduces the rate of restenosis. However, rarely is stenting considered due to the lack of adequate commercially available products. There are currently no good commercially available options for short or long term stenting of the entire frontal sinus cavity following sinus surgery.

Frontal stenting is currently considered for failed attempts at keeping the frontal sinus pathway open. One commercially available frontal sinus stent is the “Rains stent”. The Rains stent is described in U.S. Pat. No. 5,693,065 and includes a short silicone rubber tube with an egg-shaped bulb on one end. Like many other stents, the limitations of the Rains bulbous stent is inserted endoscopically into one side of the sinus area.

Rains indicates that his frontal sinus stent is self-retaining. However, the Rains bulbous stent is very inflexible and due to the relatively inflexible structure of the stent, it causes trauma to the delicate area that is to be stented, and results in a lower patency rate.

Additionally, due to the very complex anatomy of the frontal sinus cavity, the Rains stent is also relatively large and difficult to place. The Rains stent like many other stents are restricted by their structure and allowable function for placement within the frontal sinus.

Frontal sinus stents do not deliver therapeutic substances and are for that reason alone frequently used concurrently with orally administered drugs or topical nasal sprays. Accordingly, the field of frontal sinus surgery suffers from a lack of a frontal sinus stent that may be releasably secured in all areas of the frontal sinus drainage pathway. Such devices and methods are needed in order to maintain full patency of all areas of the frontal sinus cavities and drainage pathway during the healing process following sinus surgery while optionally applying therapeutic drugs.

### SUMMARY OF THE INVENTION

An aspect of the invention provides for a bilateral frontal sinus device having an elongate horizontal tube; an elongate vertical tube; and an interstitial member adjacent the horizontal tube and vertical tube therebetween. In an embodiment, the elongate horizontal tube and elongate vertical tube are flexible.

Another aspect of the invention provides for a bilateral frontal sinus device including an expandable horizontal tube; an expandable vertical tube attached to the horizontal tube; an interstitial member joining the horizontal tube and vertical tube therebetween for opening and closing the expandable horizontal tube. In another embodiment of the invention the expandable horizontal tube is configured to receive, retain, disperse, or a combination thereof of a medicated or non-medicated substance.

A further aspect of the invention provides for a bilateral frontal sinus device having a flexible elongate horizontal tube; a flexible elongate vertical tube; and an interstitial member adjacent the horizontal tube and vertical tube therebetween, wherein the interstitial member is flexible for movement of arms of the horizontal tube from 1-10 degrees through to 180 degrees. In an embodiment of the present invention there is provided a syringe attachment member releasably secured to a proximate end of the elongate vertical tube. A further embodiment includes a retractable tubular insert, for movement within the vertical tube, made from a rigid material thereby allowing for either movement therein or fixed therein for manual draining of fluids.

Another aspect of the invention provides for a substance delivering bilateral sinus system for delivering a substance to a bilateral frontal sinus cavity, the system including (a) a delivery guide configured to extend through a nostril to position a distal end of the guide in or near the bilateral frontal sinus cavity while a proximal end of the guide is located outside of the nostril; (b) an elongate substance delivery catheter having a proximal end and a distal end; and (c) a substance delivering frontal sinus device extending from and in fluid communication with the distal end of the delivery catheter. At least a portion of the device is configured to fit within both sides of the bilateral sinus cavity, wherein the device includes (i) an expandable elongate horizontal tube, (ii) an expandable elongate vertical tube attached to the horizontal tube, wherein the expandable horizontal tube is configured to receive, hold, or disperse a substance after the device has been positioned in the bilateral sinus cavity, and (iii) a flexible interstitial member joining the horizontal tube and vertical tube therebetween for vertical and horizontal movement from 1-10 degrees through to 180 degrees. In an embodiment of the invention, the device is configured to receive, hold, disperse, or a combination of a medicated or non-medicated substance during and/or after the device has been positioned in the bilateral sinus cavity. In another embodiment, the flexible interstitial member joining the horizontal tube and vertical tube therebetween is used for movement between an open position and a closed position. An embodiment of the invention provides for the syringe attachment member releasably secured to a proximate end of the elongate vertical tube or a retractable tubular insert for movement within the vertical tube. The tubing of the device is made from a silicon based rubber and the retractable tubular insert is made of a harder material than the silicon rubber.

BRIEF DESCRIPTION OF THE DRAWING

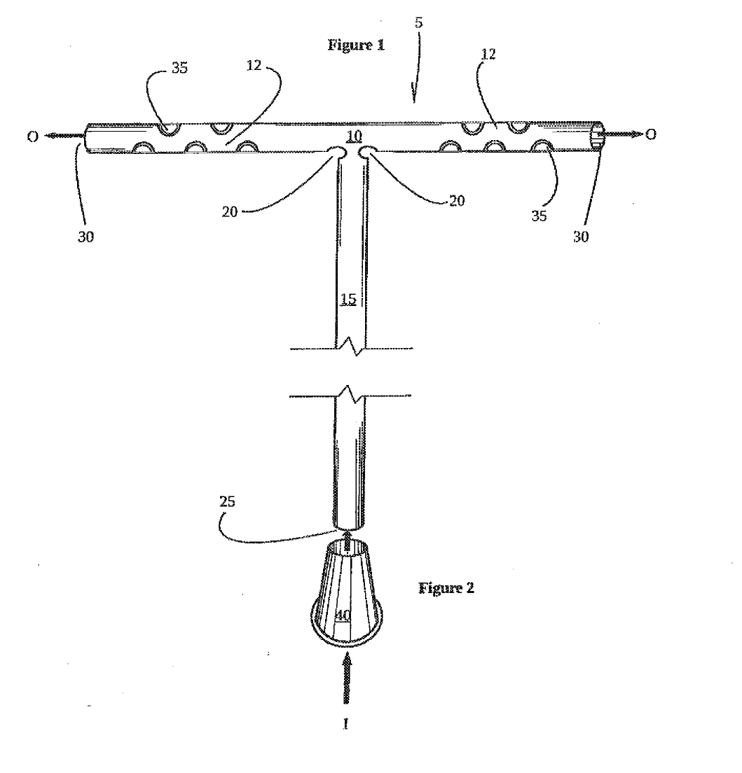

FIG. 1 is a frontal view of bilateral frontal nasal stent in an open position.

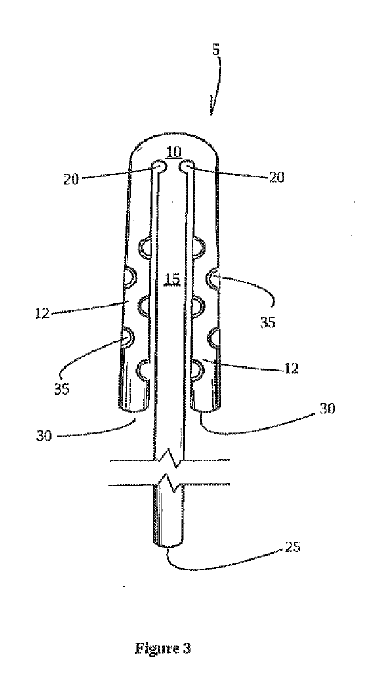

FIG. 2 is a frontal view of an adaptor used in conjunction with the embodiment of FIG. 1.

FIG. 3 is a frontal view of bilateral frontal nasal stent of FIG. 1 in a closed position.

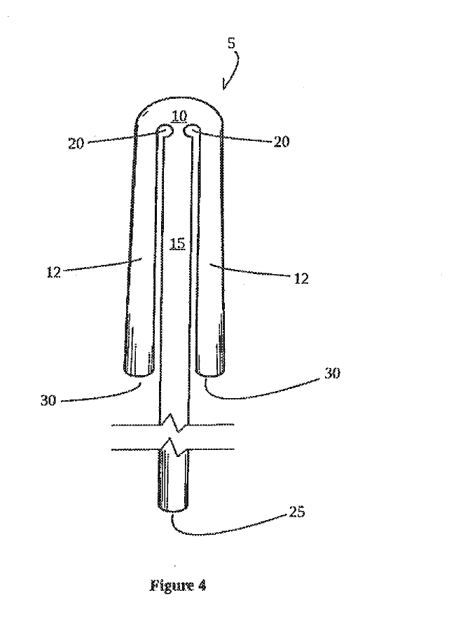

FIG. 4 is a frontal view of a further embodiment of the present invention in a closed position.

### DETAILED DESCRIPTION OF THE INVENTION

Referring to FIG. 1, a bilateral frontal sinus device 5 is shown in an open position providing a tubular structure of a predetermined length of horizontal tube 10 defined as arms 12 adjoined a predetermined length of vertical tube 15. The horizontal and vertical tubes 10, 15 are adjoined at a point along the length of each tube defined by a position of an interstitial member 20 therebetween. The term “adjoined” will be construed to include integral formations as well as connections of separate parts.

The vertical tube 15 receives at least one substance from source I through opening or aperture 25 and out O via the arms 12 of horizontal tube 10, through at least one primary aperture 30, secondary aperture 35 or a combination of both depending on the chosen area of the bilateral sinus to target. The tubular structure of the bilateral frontal sinus device 5 when in an open position resembles a T-shaped structure, however, to fit a specific anatomy of a patient this shape can vary according to a predetermined position of the interstitial member 20 and length of the horizontal and vertical tubes 10, 15. The term “apertures” will be construed to include integrally defined formations as well as other defined openings.

The length of the vertical tube 15 can vary from about 10 cm to about 20 cm, dependent on factors such as age of the patient and their respective morphology as it relates to the use of the tubes 10, 15. The length of the horizontal tube 10 can also vary from about 2 cm to about 8 cm. The the inside diameter of the tubes 10, 15, can vary from about 0.1 mm to about 2.0 mm, whereas the outside diameter of the tubes 10, 15, can also vary from about 1.0 mm to about 5.0 mm. The primary aperture 30, has a circumference and diameter larger or smaller that the circumference and diameter of the secondary apertures 35 and can vary from about 0.1 cm to about 2.0 cm.

Dimensions of the interstitial structure 20 can also vary in accordance with the circumference and diameter of tubes 10, 15 based on the applied location and use of the device 5. The interstitial structure 20 can be formed providing an aperture in between the vertical tube 15, and horizontal tube 10. Interstitial member 20 can—without forming an aperture—have an inside and outside diameter less than the inside and outside diameters of tubes 10, 15.

The purpose of the interstitial structure 20 is to allow flexible open and closed movement of the arms 12 of horizontal tube 10 having a degree of freedom from about 1 degree to about 180 degrees as defined by movement of arm 12 through an arc, for example, when moved from a closed to open position.

The ease of insertion of device 5 is aided by the amount of flexibility of horizontal tube 10, from a closed position FIGS. 3 and 4 to and open position as shown in FIG. 1. Upon removal device 5 arms 12 of the horizontal tube 10 are raised about the interstitial member 20 for ease of removal. For example, when in the released position, the device 5 resembles a close-set two pronged fork shaped structure (not shown).

In FIG. 2, a syringe connector 40 can be releasably attached to a proximate end of the vertical tube 15 or be permanently fixed thereto. The syringe connector 40 can connect to a retractable tubular insert(not shown) having an outside diameter less than the inside diameter of the vertical tube 15, for ease of movement within the vertical tube 15. The retractable tubular insert can be moved within the vertical tube 15 to remain out of sight until the need for attaching to the syringe connector 40 arises. The retractable tubular insert is made of a material more rigid than the silicon vertical tube 15.

FIG. 3 shows the bilateral device 5 in a closed position with the horizontal tube 10 forced downward alongside the vertical tube 15. In this closed position, the bilateral device 5 is inserted in to a delivery guide (not shown) that is configured to extend through a nostril to position a distal end of the guide in or near the frontal sinus cavity whereupon the device 5 is released from the delivery guide allowing for upward movement, via open movement of the interstitial member 20, of the horizontal tubing 10 to move from a closed position (FIGS. 3 and 4) of about 1-10 degrees through to an angle of about 90 degrees into a right angled open position (FIG. 1) within both sides of the sinus cavity(not shown).

Referring to FIG. 4, the bilateral device 5 is in a closed position with the horizontal tube 10, along side the vertical tube 15, absent secondary apertures 35. The vertical tube 15 and horizontal tube 10 can be adjusted to accommodate the specific needs of the recipient and for the particular use. In the open position the T-shaped structure of the bilateral device 5 is retained within the sinus cavity, thereby allowing for bilateral movement of medication and/or irrigation fluid, into both of the sinus cavities at the same time while maintaining the integrity of the opened bilateral sinuses.

The interstitial member 20 adjoined the vertical tube 15 and horizontal tube 10 is flexed for movement from a closed position to an open position and to a released position in which arms 12 are moved from one degree of freedom to another defined by movement of the arm 12 through an arc of from about 1 degrees to about 180 degrees. The interstitial member 20, when not defined as an opening and defined by a thin layer of silicon can also provide a secondary role as a narrowing of the vertical and horizontal tubes 10, 15, thereby limiting a back flow of injected substance both vertically and horizontally. Furthermore, end user of the device 5 can control the rate of fluid leaving the vertical tube 15 by gently pulling in a downward manner on the vertical tube 15 to widen the gap between the interstitial member 20 to control an amount of irrigation fluid return. The retractable tubular insert may be used to aid in the control of the fluid flow.

Although the invention has been described with reference to specific embodiments, this description is not meant to be construed in a limited sense. Various modifications of the disclosed embodiments, as well as alternative embodiments of the inventions, will become apparent to persons skilled in the art upon reference to the description of the invention. It is, therefore, contemplated that the appended claims will cover such modifications that fall within the scope of the invention.

What I claim is:

1. A bilateral frontal sinus device comprising:
an elongate horizontal tube;
an elongate vertical tube; and
an interstitial member adjoined the horizontal tube and vertical tube therebetween.

2. The bilateral device of claim 1, wherein the elongate horizontal tube and the elongate vertical tube are flexible.

3. The bilateral device of claim 1, wherein the elongate horizontal tube and the elongate vertical are expandable.

4. The bilateral device of claim 1, wherein the length of elongate horizontal tube is from about 2 cm to about 10 cm and the length of the elongate vertical tube is from about 10 cm to about 20 cm.

5. The bilateral device of claim 4, wherein the length of elongate horizontal tube is from about 3 cm to about 5 cm and the length of the elongate vertical tube is from about 12 cm to about 15 cm.

6. The bilateral device of claim 1, wherein an inside diameter of the vertical tube or horizontal tube is from about 0.1 mm to about 2.0 mm and an outside diameter of the vertical tube or horizontal tube is from about 1.0 mm to about 5.0 mm.

7. The bilateral device of claim 1, wherein the vertical tube, receive, retain, or disperse medicated or non-medicated substance.

8. The bilateral device of claim 1, wherein arms of the horizontal tube, receive, retain, and disperse medicated or non-medicated substance.

9. The bilateral device of claim 1, wherein the interstitial member has an inside diameter and outside diameter less than the inside diameter and outside diameter of the horizontal and vertical tubes.

10. The bilateral device of claim 1, wherein the interstitial member is defined by the structure of an aperture.

11. The bilateral device of claim 1, wherein the interstitial member moves or flexes between 1-10 degrees through to 180 degrees.

12. The bilateral device of claim 1, wherein the interstitial member adjacent the horizontal tube and vertical tube is flexed for movement from a closed position to an open position and a released position.

13. The bilateral device of claim 1, wherein the tubing is made from a silicon based rubber.

14. The bilateral device of claim 1, wherein a syringe attachment member is releasably secured to a proximate end of the elongate vertical tube.

15. The bilateral device of claim 1, wherein a syringe attachment member is releasably secured to a proximate end of the elongate vertical tube.

16. A substance delivering bilateral sinus system for delivering a substance to a bilateral frontal sinus cavity, the system comprising:
(a) a delivery guide configured to extend through a nostril to position a distal end of the guide in or near the bilateral frontal sinus cavity while a proximal end of the guide is located outside of the nostril;
(b) an elongate substance delivery catheter having a proximal end and a distal end; and
(c) a substance delivering frontal sinus device extending from and in fluid communication with the distal end of the delivery catheter, wherein at least a portion of the device is configured to fit within both sides of the bilateral sinus cavity, wherein the device comprises:
(i) an expandable elongate horizontal tube,
(ii) an expandable elongate vertical tube attached to the horizontal tube, wherein arms of the horizontal tube are configured to receive, hold, or disperse a substance after the device has been positioned in the bilateral sinus cavity, and
(iii) a flexible interstitial member adjoining the horizontal tube and vertical tube therebetween for vertical and horizontal movement from about 1-10 degrees through to about 180 degrees.

17. A substance delivering bilateral sinus system for delivering a substance to a bilateral frontal sinus cavity, the system comprising:
(a) a delivery guide configured to extend through a nostril to position a distal end of the guide in or near the bilateral frontal sinus cavity while a proximal end of the guide is located outside of the nostril;
(b) an elongate substance delivery catheter having a proximal end and a distal end; and
(c) a substance delivering a frontal sinus device extending from and in fluid communication with the distal end of the delivery catheter,
wherein at least a portion of the device is configured to fit within both sides of the bilateral sinus cavity, wherein the device comprises:
(i) a horizontal tube;
(ii) a vertical tube adjoined to the horizontal tube, wherein the device is configured to receive, hold, disperse, or a combination of a medicated or non-medicated substance during and/or after the device has been positioned in the bilateral sinus cavity;
(iii) a flexible interstitial member adjoined the horizontal tube and vertical tube therebetween for movement between an open position, a closed position, released position; and
(iv) a syringe attachment member releasably secured to a proximate end of the elongate vertical tube or a retractable tubular insert for movement within the vertical tube.
